(19) United States
(12) Patent Application Publication
(10) Pub. No.: US 2013/0302907 A1
(43) Pub. Date: Nov. 14, 2013

(54) METHOD OF ASSAYING ANTIGEN AND REAGENT THEREFOR

(71) Applicant: DENKA SEIKEN CO., LTD., Tokyo (JP)

(72) Inventors: Shosaku MOTODA, Niigata (JP); Yasunori MINAKAWA, Niigata (JP)

(73) Assignee: DENKA SEIKEN CO., LTD., Tokyo (JP)

(21) Appl. No.: 13/948,052

(22) Filed: Jul. 22, 2013

Related U.S. Application Data
(63) Continuation of application No. 12/967,989, filed on Dec. 14, 2010, now abandoned.
Application No. 12/967,989 is a continuation of application No. 12/709,394, filed on Feb. 19, 2010, now abandoned.
Application No. 12/709,394 is a continuation of application No. 11/661,493, filed on Feb. 27, 2007, now abandoned.

(30) Foreign Application Priority Data
Aug. 31, 2004 (JP) 2004-251456

(51) Int. Cl. G01N 33/543

(52) U.S. Cl. CPC G01N 33/54313
USPC 436/501

(57) ABSTRACT

A latex agglutination method by which the measurement range is extended and the sensitivity of the measurement in the low concentration range is increased, is disclosed. The method for measuring a test antigen by latex agglutination uses two types of large and small particles, having different average particle sizes. Each latex particle is sensitized with an antibody which undergoes antigen-antibody reaction with the test antigen. The purity of the antibody immobilized on the latex particles is within a specific range. The ratio of the amount of the antibody immobilized per one small latex particle to the amount of the antibody immobilized per one large latex particle; the average particle size of the large latex particles; the average particle size of the small latex particles; the concentration of the large sensitized latex particles in the antigen-antibody reaction system; and the concentration of the small sensitized latex particles in the reaction system are within a specific range. The large sensitized latex particles and the small sensitized latex particles are reacted with the test antigen in the state suspended in a buffer, and then the agglutination of the sensitized latex particles is optically measured.

This application is a Continuation application of co-pending U.S. application Ser. No. 12/967,989 filed on Dec. 14, 2010, which is a Continuation of application Ser. No. 12/709,394, now abandoned, filed on Feb. 19, 2010, which is a Continuation of application Ser. No. 11/661,493 now abandoned, filed on Feb. 27, 2007, which is the National Phase under 35 U.S.C. §371 of PCT International Application No. PCT/JP2005/015788 which has an International filing date of Aug. 30, 2005, and which claims priority to Japanese Application No. 2004-251456 filed on Aug. 31, 2004, the entire contents of each application are hereby incorporated by reference.

### TECHNICAL FIELD

The present invention relates to a method for measuring an antigen by latex agglutination method and a reagent therefor.

### BACKGROUND ART

Immunoassays utilizing latex particles are applied to clinical tests for quantifying antigenic substances contained in body fluids such as serum, blood plasma and urine. Since they enable simple and rapid measurements by using automatic analyzers, they are widely used.

In recent years, applied techniques aiming at further promoting measurement performance were proposed. For example, by the reagent employing two types of latex particles having different average particle sizes on which the same antibody is immobilized in at least two different amounts (Patent Literature 1) or by the measuring method using two or more types of latex particles having different average particle sizes, sensitized with an antibody (Patent Literature 2), measurements in a wide range may be attained. By the method in which a polyclonal antibody and a monoclonal antibody are immobilized on insoluble carrier particles having a single average particle size, the same effect may be obtained (Patent Literature 3). Further, measuring methods in which particles having a small particle size coated with an antibody with a low reactivity and particles having a large particle size coated with an antibody with a high reactivity are used (Patent Literatures 4 and 5) have been proposed. Still further, Non-patent Literatures 1 and 2 disclose immunoassays by agglutination method, which use a mixed sensitized particles using two types of large and small carrier particles, wherein a monoclonal antibody having a low reaction rate is immobilized on the carrier particles having a smaller average particle size and a monoclonal antibody having a high reaction rate is immobilized on the carrier particles having a larger average particle size.

However, the methods described in these literatures have a problem in that the concentration range in which measurement may be attained is narrow, and the measurement in the high concentration region is difficult.

Patent Literature 1: Japanese Patent Publication (Kokoku) No. 63-14783

Patent Literature 2: Japanese Patent Publication No. 2588174

Patent Literature 3: Japanese Laid-open Patent Application (Kokai) No. 10-90268

Patent Literature 4: Japanese Laid-open Patent Application (Kokai) No. 11-108929

Patent Literature 5: Japanese Patent Publication (Kokai) No. 3513075

Non-patent Literature 1: Journal of Clinical Laboratory Analysis 12:137-144 (1988)

Non-patent Literature 2: Japanese Journal of Medicine and Pharmaceutical Science, 42(5):781-788,1999

### DISCLOSURE OF THE INVENTION

### Problems which the Invention Tries to Solve

In many of the inspection items tested in clinical immunoserological tests, the point important for making a clinical judgment is located in the low concentration range. Thus, it is apparent that the accuracy of measurement in the low concentration range largely influence the test results. Although by the techniques employing a plurality of types of latex particles having different particle sizes, a larger measurement range is attained than that attained in the methods employing a single type of particles, and measurement in the low concentration range may also be attained, as described in Patent Literatures 2, 4 and 5, further promotion of the accuracy of measurement is desired. For the promotion of the accuracy of measurement, increase in the sensitivity of the measurement system is effective. However, in the prior art, increase in the sensitivity of the measurement system accompanies reduction of the measurement range as described in Patent Literature 2. By the methods described in Non-patent Literatures 1 and 2, measurement in the high concentration range is difficult, and the measurement range is narrow.

Accordingly, an object of the present invention is to provide a latex agglutination method by which the measurement range is extended and the measurement sensitivity in the low concentration range is promoted.

### Means for Solving the Problems

The present inventors intensively studied to discover that the measurement sensitivity in the low concentration range may be increased and the measurement range in the high concentration range may be extended by using two types of large and small latex particles, that is, a mixture of latex particles having a large average particle size (large latex particles) sensitized with (carrying) an antibody in an amount at which the maximum reaction rate is attained or in an amount close thereto, and latex particles having a small average particle size (small latex particles) sensitized with the antibody in an amount smaller than the usual amount in order to decrease the reaction rate, which mixture has the optimized average particle sizes of the large and small particles, and by optimizing the concentrations of the particles in the reaction system, thereby completing the present invention.

That is, the present invention provides a method for measuring a test antigen by latex agglutination using sensitized latex particles, the method comprising reacting sensitized latex particles suspended in a buffer with the test antigen; the sensitized latex particles being two types of large and small particles, having different average particle sizes, each of the latex particles being sensitized with an antibody or an antigen-binding fragment thereof, which antibody undergoes antigen-antibody reaction with the test antigen; the antibody or antigen-binding fragment thereof immobilized on the latex particles having a purity of not less than 14% by weight based on the total amount of proteins carried on the particles; the amount of the antibody or antigen-binding fragment thereof immobilized per one particle of the small particles is 0.5 to 10% by weight of the amount of the antibody or antigen-binding fragment thereof immobilized per one particle of the large particles; the large latex particles having an average particle size of 0.15 to 0.29 μm; the small latex particles having an average particle size of 0.05 μm to 0.10 μm; the concentration of the large latex particles in the antigen-antibody reaction system being 0.01 to 0.04 w/v %; the concentration of the small latex particles in the antigen-antibody reaction system being 0.05 to 0.2 w/v %; and optically measuring agglutination of the sensitized latex particles. The present invention also provides a reagent for measuring an antigen by latex agglutination method, comprising two types of large and small particles, having different average particle sizes, in a buffer; each of the latex particles being sensitized with an antibody or an antigen-binding fragment thereof, which antibody undergoes antigen-antibody reaction with the test antigen; the antibody or antigen-binding fragment thereof immobilized on the latex particles having a purity of not less than 14% by weight based on the total amount of proteins carried on the particles; the amount of the antibody or antigen-binding fragment thereof immobilized per one particle of the small particles is 0.5 to 10% by weight of the amount of the antibody or antigen-binding fragment thereof immobilized per one particle of the large particles; the large latex particles having an average particle size of 0.15 to 0.29 μm; the small latex particles having an average particle size of 0.05 μm to 0.10 μm.

### Effects of the Invention

By the present invention, an immunoassay as well as a reagent therefor was established, by which measurement with high sensitivity in the low concentration range and extension of the measurement range in the high concentration range may be attained, and accuracy of measurement in the low concentration range, which is important for making a clinical judgment in immunoserological tests or the like is increased so that more accurate judgment may be attained.

### BRIEF DESCRIPTION OF THE DRAWINGS

FIG. 1 comparatively shows the relationships between the CRP level and the amount of change in absorbance, which were obtained using Reagent 1, 2 and 3 prepared in Example, respectively.

FIG. 2A shows the relationship between the CRP level and the amount of change in absorbance, which was obtained using Reagent 4 prepared in Example.

FIG. 2B is an enlarged view showing the low CRP concentration range in FIG. 2A.

FIG. 3A comparatively shows the relationships between the CRP level and the amount of change in absorbance, which were obtained using the reagent according to the present invention and a reagent according to Comparative Example, respectively.

FIG. 3B is an enlarged view showing the low CRP concentration range in FIG. 3A.

FIG. 4 shows the relationship between adsorption isotherm and the maximum amount of adsorption on the adsorption 100% line.

### BEST MODE FOR CARRYING OUT THE INVENTION

The latex particles used in the method of the present invention are sensitized with (i.e., carry) an antibody which undergoes antigen-antibody reaction with the antigen to be measured (hereinafter referred to as “specific antibody”) or an antigen-binding fragment thereof. The specific antibody may be either a polyclonal antibody or a monoclonal antibody. The particles may be sensitized with, instead of the antibody, a fragment of the antibody, having a binding ability with the corresponding antigen, such as Fab fragment or F(ab′)₂ fragment (referred to as “antigen-binding fragment” in the present description and claims).

The purity of the antibody or the antigen-binding fragment thereof immobilized on the latex particles is not less than 14% by weight, preferably not less than 16% by weight based on the amount of proteins carried on the particles. If the purity is less than 14% by weight, sufficient measurement sensitivity in the low concentration range is not obtained. For example, a polyclonal antibody is usually prepared by injecting an antigen to an animal; collecting the blood from the animal after a prescribed time has passed; and subjecting the collected blood as a starting material to affinity chromatography using a Protein A column or the like. Thus, the polyclonal antibody contains other immunoglobulins in addition to the specific antibody which undergoes antigen-antibody reaction with the antigen. In the present invention, the percentage (purity) of the amount of the specific antibody (mg/mL) based on the amount of the total proteins (mg/mL) in the antibody solution is important, and an antibody (antibody solution) containing the specific antibody in an amount of not less than 14% by weight may preferably be used. As for a monoclonal antibody, an antibody (antibody solution) obtained by purifying ascites or culture medium containing the specific antibody as a starting material, which was obtained in growing the hybridomas producing the antibody in abdominal cavity of a mouse or in a culturing apparatus, may be used. A monoclonal antibody (antibody solution) containing the specific antibody in an amount (mg/mL) of not less than 14% by weight based on the total amount (mg/mL) of proteins may preferably be used as in the case of the polyclonal antibody.

The amount of the total proteins (mg/mL) in the antibody solution may be measured by a known method in which absorbance at a wavelength of 280 nm is measured. The amount of the specific antibody may be measured by the conventional Becker's method (Experimental Methods A in Immunology, pp 164-171, 1976, published by Japanese Society for Immunology; Original Article: Becker W.: Immunochemistry, 6: 539-546, 1969).

In the method of the present invention, two types of large and small particles, having different average particle sizes, are used. The latex particles having the larger average particle size (large latex particles) have an average particle size (diameter) of 0.15 to 0.29 μ, preferably 0.20 to 0.29 μ. If the average particle size of the large latex particles is within this range, a high measurement sensitivity is attained in the low concentration range. On the other hand, the average particle size (diameter) of the latex particles having the smaller average particle size (small latex particles) is 0.05 to 0.10 μ, preferably 0.06 to 0.08 μ. If the average particle size of the small latex particles is within this range, an accurate measurement in the high concentration range may be attained, so that the concentration range in which measurement may be attained is extended to the side of high concentration.

As the latex particles, those particles which are conventionally and widely used, such as, for example, those made of a polystyrene, styrene-butadiene copolymer, styrene-styrene sulfonate copolymer, may be employed.

The above-described large and small latex particles are sensitized with the same specific antibody or the same antigen-binding fragment thereof. Sensitization of the latex particles with the specific antibody or the antigen-binding fragment thereof may be carried out by a conventional well-known method. For example, sensitization may be attained by suspending the latex particles in a solution of the specific antibody or the antigen-binding fragment thereof and stirring the suspension so as to physically adsorb the specific antibody or the antigen-binding fragment thereof.

In the present invention, the amount (average) of the antibody or antigen-binding fragment thereof immobilized per one particle of the small particles is 0.5 to 10% by weight of the amount (average) of the antibody or antigen-binding fragment thereof immobilized per one particle of the large particles. Since the reaction rate may be decreased by decreasing the amount of the antibody immobilized on the latex particles, the optimum amount of the antibody may be selected from the measured time course of the reaction. By so doing, the calibration curve may be extended to the side of the high concentration range, and the concentration range in which measurement may be attained is extended to the side of high concentration. The amount of the antibody or antigen-binding fragment thereof immobilized on the particles may be freely adjusted by diluting the specific antibody or antigen-binding fragment thereof with a buffer solution. The optimum amount of sensitization within the above-described range may appropriately be selected depending on the type of the antigen to be measured, purpose of measurement, measurement range, antibody titer and so on (see Test Examples below).

On the other hand, the larger the amount of the antibody adsorbed on the latex particles, the higher the reaction rate, and the larger the particle size, the higher the reaction rate. Thus, by sensitizing the large latex particles with the antibody or antigen-binding fragment thereof in an amount as large as possible, the measurement sensitivity in the low concentration range is increased. Therefore, the amount of the specific antibody or antigen-binding fragment thereof immobilized on the large latex particles is preferably as much as possible. Therefore, the amount of 85% to 120%, more preferably 95% to 120% of the maximum adsorption amount on adsorption isotherm 100% line is preferred. However, the amount of the specific antibody or antigen-binding fragment thereof may appropriately be selected depending on the type of the antigen to be measured, purpose of measurement, measurement range, antibody titer and so on. The maximum adsorption amount on adsorption isotherm 100% line may easily be determined based on the concept of adsorption isotherm well-known in the art of surface chemistry. This method is described in, for example, Microparticle Reagent Optimization, Seradyn, Inc. 1994, and will be described below in detail.

The maximum adsorption amount on the 100% line shown by adsorption isotherm (adsorption isotherm 100% line) may concretely be determined as follows: That is, the amount of protein (μg) per 1 mg of latex particles, which protein was added during the sensitization step of the latex particles, is taken along the X-axis, and the amount of the adsorbed protein (μg) per 1 mg of latex particles, which was measured after sensitization is taken along the Y-axis, thereby preparing an adsorption isotherm. Then a straight line (adsorption 100% line) of Y=X is drawn, and the maximum adsorption on the adsorption 100% line is determined. To more comprehensively explain these, a graph is shown in FIG. 4. The amount of the adsorbed protein (μg) per 1 mg of the latex particles is determined by measuring the amount of protein (μg) after filtration of the centrifugation supernatant after sensitization through a 0.1 μm filter unit (ULTRAFREE-MC, Millipore), and subtracting the thus measured amount from the amount of protein (μg) before sensitization. The adsorption ratio (%) is calculated according to the following equation:

$$Adsorption Ratio ( \% ) = \frac{Amount of Adsorbed Protein ( µ g )}{Protein Amount before Sensitization ( µ g )}$$

In the method of the present invention, the concentration of the large latex particles in the reaction system is 0.01 to 0.04 w/v %, and the concentration of the small latex particles in the reaction system is 0.05 to 0.2 w/v %. If the concentrations of the large and small latex particles are within these ranges, highly sensitive measurement in the low concentration range and extension of measurement range in the high concentration range may be better attained.

Latex agglutination method per se may be carried out in exactly the same manner as in the conventional method. That is, latex agglutination method may be carried out by mixing a test sample and a suspension of the above-described large and small sensitized latex particles in a buffer, and optically measuring the agglutination caused by the antigen-antibody reaction. The optical measurement may be carried out as follows: For example, when the latex particles sensitized with the antibody and a test sample are mixed in a cell made of plastics or glass, if the antigenic substance to be measured exists in the test sample, the latex particles sensitized with the antibody agglutinate due to the antigen-antibody reaction. At that time, by radiating a light from the outside, which has a suitable wavelength selected from the range of 0.3 μm to 2.4 μand measuring the change in absorbance, the target antigenic substance existing in the test sample may be measured. The change in absorbance may be measured by measuring the change in absorbance between 60 seconds and 90 seconds from the beginning of the antigen-antibody reaction. However, the measurement time is not restricted to this range, and the target antigenic substance may be measured by measuring the change in absorbance or the like during an arbitrary time period within a relatively early stage of the reaction. Since the rate of agglutination correlates with the antigen level in the test sample, the antigen level in a test sample may be determined by applying the measured agglutination rate to a calibration curve which was preliminarily prepared by subjecting some standard solutions having known antigen levels, respectively, to the reaction and by measuring the agglutination rate. The reaction temperature is not restricted, and a temperature of about 37° C. is usually preferred.

The antigen which may be measured by the method of the present invention is not restricted at all, and any antigen whose corresponding antibody is able to be prepared may be measured by the method of the present invention. Examples of the antigen include proteins serving as a disease marker such as C-reactive protein (CRP), and pathogens such as bacteria and viruses, but the antigen is not restricted thereto. The test sample is also not restricted at all, and examples thereof include, but not limited to, body fluids such as serum, blood plasma, urine and throat swab; culture media; foods and beverages and extracts thereof In cases where it is difficult to attain an accurate measurement when a body fluid or the like is applied to the measurement as it is because the antigen level is too high, needless to say, a dilution of the body fluid or the like diluted with physiological saline may be used as the test sample.

The present invention further provides a reagent to be used in the above-described method according to the present invention. That is, the present invention also provides a reagent for measuring an antigen by latex agglutination method, comprising two types of large and small particles, having different average particle sizes, in a buffer; each of the latex particles being sensitized with an antibody or an antigen-binding fragment thereof, which antibody undergoes antigen-antibody reaction with the test antigen; the antibody or antigen-binding fragment thereof immobilized on the latex particle having a purity of not less 14% by weight based on the total amount of proteins carried on the particle; the amount of the antibody or antigen-binding fragment thereof immobilized per one particle of the small particles being 0.5 to 10% by weight of the amount of the antibody or antigen-binding fragment thereof immobilized per one particle of the large particles; the large latex particles having an average particle size of 0.15 to 0.29 μm; the small latex particles having an average particle size of 0.05 μm to 0.10 μm.

By using the small sensitized latex particles whose reaction rate is small, the present invention enables to effectively capture the antigenic substance in the test sample depending on the concentration thereof, thereby enhancing the accuracy of the measurement. On the other hand, in cases where the concentration of the antigenic substance in the test sample is very small, the large sensitized latex particles preferentially capture the antigenic substance to cause agglutination, so that a small degree of reaction may be detected as a large optical change. In cases where the concentration of the antigenic substance in the test sample is high, the antibody on the large latex particles whose reaction rate is high is rapidly consumed and the large sensitized latex particles instantly agglutinate. On the other hand, the small sensitized latex particles whose reaction rate is low keep slow reaction and the agglutination reaction of the small sensitized latex particles is kept, so that the reaction may be continued while reducing the optical change. According to the present invention, the measurement range is extended by these effects, and the measurement in the low concentration range may be attained with a higher sensitivity. Further, according to the present invention, by optimizing the average particle size of the large and small latex particles carrying the optimum amount of the antibody, and by optimizing the ratio of concentrations thereof in the reaction system, extension of the measurement range and the increase in the sensitivity of the measurement in the low concentration range are especially well attained.

The present invention will now be described more concretely by way of Examples thereof. However, the present invention is not restricted to the Examples below.

### EXAMPLE 1

### Measurement of CRP

Polystyrene latex particles having a particle size of 0.28 μm and polystyrene latex particles having a particle size of 0.06 μare sensitized with an anti-CRP rabbit polyclonal antibody (an antibody solution in which the amount of the specific antibody (mg/mL) based on the total protein amount (mg/mL) was 17%), and the resulting latex particles were suspended in glycine buffer to prepare a reagent. That is, the reagent was prepared by mixing prescribed amounts of the antibody and the polystyrene latex particles in a buffer (MES 50 mM pH 6.0, MES: 2-Morpholinoethanesulfonic acid, monohydrate); sufficiently stirring the mixture at room temperature for 60 minutes, thereby carrying out sensitization; removing supernatant after centrifugation; blocking the unsensitized regions in the surface of the polystyrene latex particles with glycine buffer containing bovine serum albumin; collecting again the polystyrene latex particles sensitized with the antibody by centrifugation; and dispersing and suspending the latex particles in glycine buffer by sufficiently conducting sonication.

One type of the polystyrene latex particles with a particle size of 0.28 μm sensitized with the antibody in an amount of 120% of the maximum adsorption amount on the adsorption 100% line shown by the adsorption isotherm; and three types of polystyrene latex particles with a particle size of 0.06 μm sensitized with the antibody in an amount of 2.29, 1.61 or 1.45% by weight of the amount of the antibody immobilized per one particle of the polystyrene latex particles with a particle size of 0.28 μm were prepared.

The amount of the immobilized antibody was concretely controlled by carrying out the sensitization after preliminarily diluting the antibody in a buffer (MES, 50 mM, pH6.0) to a concentration suited for the desired amount of the antibody to be immobilized.

### Reagents Constituted by Each Type of Particles Before Mixing

As examples of the present invention, suspensions of the above-described particles with a particle size of 0.06 μm sensitized with the antibody in amounts of 2.29, 1.61 and 1.45% by weight, respectively, suspended in glycine buffer to a particle concentration of 0.175% (w/v) were prepared and designated Reagents 1, Reagent 2 and Reagent 3, respectively. A suspension of the antibody-carrying particles with a particle size of 0.28 μsuspended in glycine buffer to a concentration of 0.04% (w/v) was prepared and designated Reagent 4.

### Reagent Constituted by Mixed Particles

A reagent according to the present invention was prepared by mixing the antibody-carrying particles with a particle size of 0.28 μand the particles (the same particles as used in Reagent 2) with a particle size of 0.06 μcarrying the antibody in an amount of 1.61% by weight in glycine buffer so as to attain particle concentrations of 0.04% (w/v) and 0.175% (w/v), respectively. As Comparative Example, a reagent prepared by the method described in Patent Literature 5 was used. As a sample for measurement, CRP standards prepared by diluting purified human CRP antigen with normal human serum were used. The concentrations of the CRP standards were in accordance with Blood Plasma Proteins International Standards Preparations CRM470. Standards having concentrations of 0.25, 0.5, 1, 2, 4, 8, 16, 32, 48, 67 and 100 mg/dL, respectively, were prepared. As a standard with a concentration of 0 mg/dL, physiological saline was used. Measurements were carried out using Automatic Analyzer for Biochemical Tests TBA-80FR (produced by TOSHIBA) under the following conditional parameters: That is, to 3 μL of the sample for measurement, 150 μL of glycine buffer as a first reagent, and 150 μL of the prepared reagent as a second regent were added, and the analyzer was set such that the change in absorbance (AABS/min) per 1 minute between the Photometry Point 21 and Photometry Point 27 after the beginning of the reaction (after adding the second reagent) was measured at a wavelength of 572 nm. ΔABS/min×10,000 was defined as the change in absorbance.

### TEST EXAMPLE 1

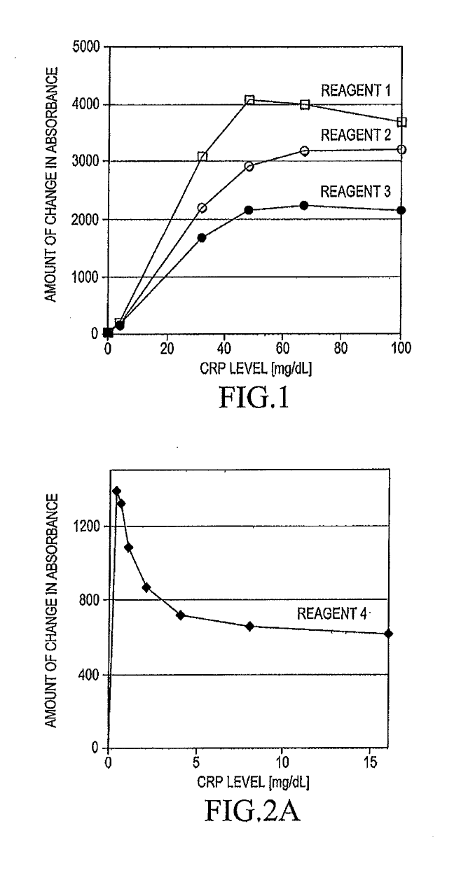

Calibration curves showing the relationships between the CRP level and the amount of change in absorbance, using Reagent 1, 2 and 3, respectively, were prepared. These are shown in FIG. 1. As shown in FIG. 1, with Reagent 1, since the reaction rate is high, the calibration curve rises up to only 48 mg/dL. With Reagent 2, since the reaction rate is lower than that with Reagent 1, the calibration curve rises up to 100 mg/dL. With Reagent 3, since the reaction rate is too low, the initial rise of the calibration curve is low. Further, since the amount of the antibody necessary for the reaction is insufficient, the calibration curve rises up to only 67 mg/dL. From these results, it can be seen that by using Reagent 2, the calibration curve rises in the high concentration range, so that accurate measurement in the high concentration range and extension of measurable concentration range to the side of the high concentration may be attained. Thus, the effect of slowing the reaction by making the amount of the antibody adsorbed to the latex particles small is evident.

### TEST EXAMPLE 2

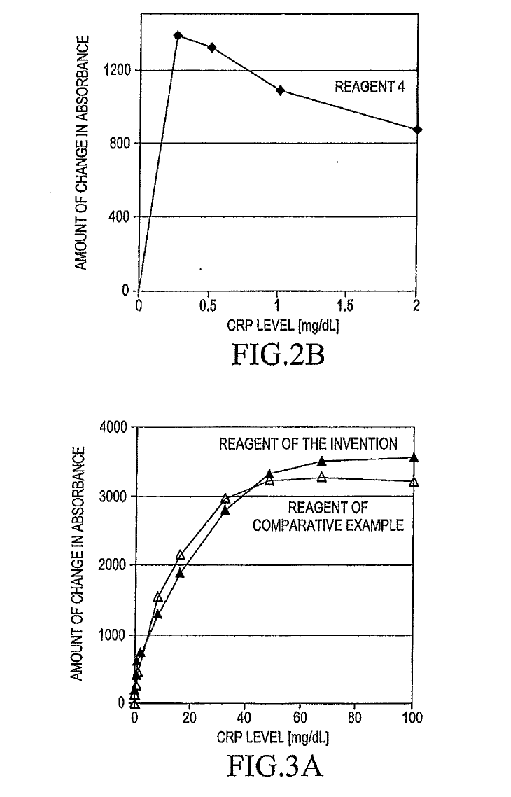

A calibration curve showing the relationship between the CRP level and the amount of change in absorbance was prepared using Reagent 4. This is shown in FIGS. 2A and 2B. FIG. 2B enlargingly shows the area of the CRP level of 0 to 2 mg/dL. The calibration curve well rises in the low concentration range between 0 and 0.25 mg/dL, so that it can be seen that the measurement sensitivity is high. However, since the antibody reacts instantly, the calibration curve does rise in the high concentration range.

### TEST EXAMPLE 3

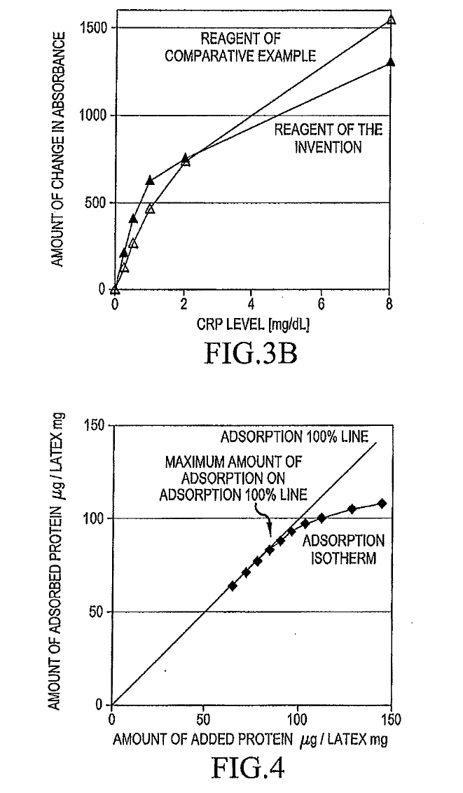

Calibration curves showing the relationships between the CRP level and the amount of change in absorbance, using the reagent according to the present invention and the reagent according to Comparative Example, respectively, were prepared. These are shown in FIGS. 3A and 3B. FIG. 3B enlargingly shows the area of the CRP level of 0 to 8 mg/dL. As shown by the results with the reagent according to the present invention, detection with higher sensitivity than that with the reagent of Comparative Example is attained in the low concentration range between 0 and 2 mg/dL (see FIG. 3B). Further, in the high concentration range, while measurement may be attained up to only 67 mg/dL with the reagent according to Comparative Example, measurement may be attained up to 100 mg/dL with the reagent according to the present invention (see FIG. 3A). As shown in FIGS. 3A and 3B, with the reagent according to the present invention, measurement may be attained with a higher sensitivity in the range of low CRP level and measurement may be attained up to higher concentration range than with the reagent according to Comparative Example. Further, by comparing the two calibration curves obtained with Reagent 2 shown in FIG. 1 and obtained with the reagent according to the present invention shown in FIG. 3A, it can be seen that with the reagent according to the present invention, the change in absorbance in the high concentration range of 67 to 100 mg/dL is larger than that with Reagent 2, so that it can be seen that mixing of the two types of the particles is also effective for amplifying the amount of change in absorbance. From these results, it is apparent that the method of the present invention using two types of large and small latex particles, having different average particle sizes, on which prescribed amounts of the same antibody are immobilized, respectively, that is, using large sensitized latex particles exhibiting high reaction rate and small sensitized latex particles exhibiting low reaction rate, is superior.

### INDUSTRIAL AVAILABILITY

By the immunoassay according to the present invention, highly sensitive measurement in the low concentration range and extension of the measurement range in the large concentration range may be attained, so that the accuracy of measurement in the low concentration range, which is important for the clinical judgment in immunoserological tests and the like, is increased and more accurate judgment may be made. Therefore, the present invention is useful in immunoassays by latex agglutination method widely used in diagnoses of various diseases and the like.

1. A method for measuring a test antigen by latex agglutination using sensitized latex particles, said method comprising:
(a) identifying an antibody, or antigen-binding fragment thereof, that reacts with a test antigen;
(b) immobilizing the antibody, or antigen-binding fragment thereof, to small latex particles having an average particle size of 0.005 μto 0.10 μ;
(c) immobilizing the antibody, or antigen-binding fragment thereof, to large latex particles having an average particle size of 0.15 to 0.29 μin an amount determined by an adsorption isotherm 100% line,
wherein the amount of antibody or antigen-binding fragment thereof immobilized per particle of the small particles being 0.5 to 10% by weight of the amount of antibody or antigen-binding fragment thereof immobilized per one particle of the large particles and wherein the antibody, or antigen-binding fragment thereof, has a purity of not less than 14% by weight based on the total amount of proteins carried on the particles;
(d) suspending the particles of (a) and the particles of (b) in a buffer;
(e) contacting the buffer of (d) with the test antigen to achieve agglutination of the latex particles, wherein the concentration of the large latex particles is 0.01 to 0.04 w/v % and the concentration of the small latex particles is 0.05 to 0.2 w/v %;
(f) optically measuring agglutination of the latex particles; and
(g) comparing the optical measurement with a standard curve showing the relationship between the optical measurement and antigen amount to obtain the concentration of the test antigen.

2. The method according to claim 1, wherein said antibody or antigen-binding fragment thereof is immobilized on said large latex particles in an amount of 85% to 120% of the maximum adsorption amount on adsorption isotherm 100% line, wherein the adsorption isotherm 100% line is the line Y=X, wherein X is the amount of protein added to the large latex particles in a sensitizing reaction and Y is the amount of protein that adsorbs to the particles, wherein Y is determined by measuring the amount of protein after filtration of a centrifugation supernatant after sensitization through a 0.1 μfilter unit, and subtracting the thus measured amount from X, and the maximum adsorption amount on the adsorption isotherm 100% line is the value of X at the point where the adsorption isotherm line deviates from the line Y=X.

3. A reagent for measuring an antigen by latex agglutination, comprising:
(a) a buffer;
(b) small latex particles having an average particle size of 0.05 μto 0.10 μm on which is immobilized an antibody, or antigen-binding fragment thereof, which binds the test antigen; and
(c) large latex particles having an average particle size of 0.15 to 0.29 μon which is immobilized an antibody, or antigen-binding fragment thereof, which binds the test antigen,
wherein the amount of antibody or antigen-binding fragment thereof immobilized per particle of the small particles is 0.5 to 10% by weight of the amount of antibody or antigen-binding fragment thereof immobilized per particle of the large particles and wherein the antibody, or antigen-binding fragment thereof, has a purity of not less than 14% by weight based on the total amount of proteins carried on the particles.

4. The reagent according to claim 3, wherein said antibody or antigen-binding fragment thereof is immobilized on said large latex particles in an amount of 85% to 120% of the maximum adsorption amount on adsorption isotherm 100% line, wherein the adsorption isotherm 100% line is the line Y=X, wherein X is the amount of protein added to the large latex particles in a sensitizing reaction and Y is the amount of protein that adsorbs to the particles, wherein Y is determined by measuring the amount of protein after filtration of a centrifugation supernatant after sensitization through a 0.1 μm filter unit, and subtracting the thus measured amount from X, and the maximum adsorption amount on the adsorption isotherm 100% line is the value of X at the point where the adsorption isotherm line deviates from the line Y=X.

5. The method according to claim 1, wherein C-reactive protein (CRP) is measured.

6. The reagent according to claim 3, wherein the antigen is C-reactive protein (CRP).
